(19) United States
(12) Patent Application Publication
(10) Pub. No.: US 2006/0251608 A1
(43) Pub. Date: Nov. 9, 2006

(54) A Method of Treatment of Aging and Wrinkled Skin and a Kit Containing Compositions for Same

(75) Inventors: Richard Wachsberg, Toronto (CA); Charles Wachsberg, Thornhill (CA)

Correspondence Address: SYNNESTVEDT LECHNER & WOODBRIDGE LLP, P O BOX 592, PRINCETON, NJ, 08542-0592, US

(73) Assignee: Rwachsberg Holdings Inc., Concord (CA)

(21) Appl. No.: 11/381,552

(22) Filed: May 4, 2006

Related U.S. Application Data
(60) Provisional application No. 60/677,369, filed on May 4, 2005.

(51) Int. Cl. A61K 8/97; A61K 38/43; A61K 31/385; A61K 35/64; A61K 33/34; A61K 33/04; A61K 33/32; A61K 36/13; A61K 36/906; A61K 36/82

(52) U.S. Cl. 424/74; 424/729; 424/756; 424/766; 424/732; 424/638; 424/702; 424/770; 424/764; 424/774; 424/539; 424/768; 424/94.1; 514/27; 514/440; 514/456; 514/566; 514/546; 514/423; 424/722; 424/641

(57) ABSTRACT

There is disclosed a method of treating aged and wrinkled skin and a kit containing compositions and instructions for such treatment. The method consists of a daily treatment regime, with two separate oral doses approximately 12 hours apart and application of a topical formulation. The oral formulations may be the same or different from each other, and each consist of one or more antioxidants, antioxidant co-promoters, anti-inflammatory, collagen synthesis promoters and optionally other substances. The topical formulation consists of an anti-wrinkle substance, such as a cream.

### RELATED APPLICATION

This application claims the benefit under Title 35, U.S.C., S.119(e) of U.S. Provisional Application No. 60/677,369 filed on May 4, 2005.

### FIELD OF THE INVENTION

The invention relates to the field of skin treatments, and specifically compositions, formulations, and treatment for oral consumption and external application by humans for treatment of aged and wrinkled skin.

### BACKGROUND OF THE INVENTION

Aged, dry and wrinkled skin may be treated by various formulations, therapies and compositions which assist the body's natural processes for building of collagen within the skin structure, to increase the suppleness of skin and reduce wrinkling. This has cosmetic benefits, in that such therapies make the user's skin more youthful in appearance. It is known, for example, that compounds in the coenzyme Q family, such as coenzyme Q-10, applied as a cream have the ability to reduce wrinkling and the effects of aging and dry skin.

### SUMMARY OF THE INVENTION

An object of the invention is to provide improved compositions, kits, and treatment regimes, for the treatment of dry, aged and wrinkled skin.

According to one aspect, the invention relates to a method for treating wrinkled, aging, and otherwise damaged skin comprising twice daily oral consumption of formulations described herein, preferably separated in time by approximately 12 hours. The first and second formulations for the two doses contain the same or different active ingredients. The method also includes the step of applying a composition for external (topical) application to the affected area of skin. The invention further relates to a kit, consisting of the formulation in topical application form and two formulations in oral dosage form, together with instructions for consumption of the two formulations separated by approximately 12 hours from each other. It will be understood that the twice daily dosage regime permits a wide range of timing, for example the two doses may be consumed between 8 and 16 hours apart. Preferably, the formulations are visually distinguished from each other, for example by different colors, shapes, sizes or any combination thereof. The first formulation may be taken in the morning and the second formulation in the evening, or vice versa, or any other suitable and convenient twice daily regime.

The first and second oral dosage formulations each comprise a combination of one or more antioxidants and antioxidant co-promoters, anti-inflammatories, vitamins, nutritionally desirable minerals, and collagen synthesis promoters preferably with a pharmaceutically acceptable carrier. The first and second formulations preferably differ from each other. The antioxidants preferably include flavenoids, but may comprise any suitable antioxidant including an antioxidant derived from a natural source. Additional preferred ingredients are vitamin E and a co-enzyme Q such as co-enzyme Q-10.

Optionally, the first oral formulation is for morning consumption and includes a preferably natural stimulant such as caffeine and the second oral formulation is for evening consumption and includes a preferably natural sedative such as chamomile or chamomile extract.

The first oral formulation preferably comprises flax seed, preferably divided as a powder or otherwise, green tea extract, DMAE, preferably as DMAE bitartrate, ginger root in powder or other divided form, alpha-lipoic acid, bioflavonoid complex, aloe vera leaf, as extract or otherwise, n-acetyle 1-carnitine, 1-carnosine, grape seed extract, quercetin, 1-proline, tumeric powder, hyaluronic, and one or more flavenoids such as red wine, pycngenol, turmeric, and/or blueberry extract. The formulation preferably also includes excipients, fillers or other inactive substances, such as cellulose, silicon dioxide, magnesium stearate, and titanium dioxide. The formulation may comprise a gelatin capsule or other suitable dosage form for oral consumption.

The second formulation preferably comprises jujub extract, magnolia bark extract, honokiol, conjugated lipoic acid, chamomile flower, royal jelly, a coenzyme Q compound such as coenzyme Q-10, an amino acid such as 1-lysine, preferably as lysine HCl, an evergreen bark extract such as pine bark extract, reservatrol and a nucleic acid such as ribonucleic acid. The formulation optionally comprises additional inactive components as described above and in a similar fashion, comprises a gelatin capsule or other suitable oral dosage form.

The topical formulation comprises an anti-wrinkle/anti-aging skin treatment, preferably a cream such as Nutrius™ Anti-Aging and Stretch-Mark Cream.

One or both of the above oral formulations may also comprise nutritional supplements such as vitamins and minerals, for example calcium, zinc, selenium and copper. The oral and topical formulations may comprise additional compounds not defined herein.

The natural substances within the oral formulations may comprise the material itself in an unprocessed form or an extract or chemical derivative of such substance. Any reference to a natural plant substance will be understood to include any such form thereof, unless specifically identified otherwise.

Conveniently, the above three formulations are provided as a kit, having therein individual containers for the first and second oral formulations and the topical formulation, together with instructions for the twice daily consumption of the oral formulation and the periodic and preferably daily topical application of the topical formulation. It is also contemplated that the kit and related treatment regime may include additional components and steps, for example a sunscreen or additional oral or topical formulations.

### EXAMPLE 1

### Formulation 1

A first oral formulation was prepared comprising swallowable gelatin capsules comprising:

| | mg/capsule |
|---|---|
| Active ingredients | |
| flax seed powder | 80 |
| green tea (at about 50% natural caffeine) | 30 |
| DMAE (as DMAE bitartrate) | 25 |
| green tea (containing about 40% catechines) | 10 |
| ginger root powder | 12.0 |
| alpha-lipoic acid | 10 |
| bioflavonoid complex | 10 |
| aloe vera leaf (Terry Labs) | 5.0 |
| n-acetyle l-carnitine | 5.0 |
| l-carnosine | 5.0 |
| grape seed PE 4:1 | 5.0 |
| quercetin | 5.0 |
| red wine (diluted 4-1) | 3.0 |
| blueberry extract (diluted 4-1) | 2.0 |
| l-proline | 2.2 |
| tumeric powder | 2.0 |
| hyaluronic acid | 1.0 |
| vitamin A (as beta-carotene) | 10000 i.u. |
| vitamin C (from ester C) | 20 |
| vitamin E (as D-α-tocopheryl succinate) | 30 i.u. |
| vitamin B-1 (as thyaminemononitrate) | 2.0 |
| vitamin B-6 (as pyroxidine HCl) | 25 |
| folic acid 400 mg | |
| potassium (as potassium chloride) | 25 |
| calcium carbonate (USP granular 38%) | 14 |
| magnesium citrate USP/NF | 12.5 |
| manganese sulfate 31.4% | 6.5 |
| Inactive Ingredients | |
| gelatin (for gelatin capsule) | |
| cellulose (granular) | 10 |
| silicon dioxide (Syloid-Flo-Gard ™ SP) | 20 |
| magnesium stearate | 10 |
| titanium dioxide | |

### EXAMPLE 2

### Formulation 2

The second oral formulation was prepared as swallowable gelatin capsules, comprising:

| | mg/capsule |
|---|---|
| Active Ingredients (203 mg) | |
| jujub extract at 10-1 dilution | 50 |
| magnolia bark extract 1.5% honokiol | 50 |
| conjugated lipoic acid | 25 |
| chamomile flower | 112 |
| royal jelly | 5.0 |
| coenzyme Q-10 | 3.0 |
| l-lysine (as lysine HCl) | 2.2 |
| pine bark extract 75% | 2.0 |
| reservatrol | 2.0 |
| RNA (ribonucleic acid) | 2.0 |
| calcium (from coral calcium) | 19 |
| zinc (as zinc picolinate) | 2.0 |
| selenium (as selenomethionine 5000 mcg/g) | 10 |
| copper (as copper oxide) | 1.25 |
| Inactive Ingredients | |
| magnesium stearate | 10.0 |
| cellulose (granular) | 200 |
| silicon dioxide (Syloid Flo-Gard ™ SP) | 20 |

### EXAMPLE 3

### Therapeutic Kit

A kit was prepared comprising capsules of the above Example 1, identified with a label instructing the user to consume one such capsule in the morning, capsules of Example 2 in a container with a label instructing the user to consume one such capsule each evening, and Nutrius™ Anti-Aging and Anti-Stretch Mark Cream. The capsules of Examples 1 and 2 and their containers were made visually distinct from each other by color patterns. The kit further comprised instructions instructing the users to consume one each of the capsules according to the above Examples 1 and 2, approximately 12 hours apart and preferably with the first capsule in the morning and the second capsule in the evening, although the order of consumption may be reversed, and to apply the cream topically to skin areas affected by aging, wrinkling, stretch-marks and other like skin conditions. The cream may be applied either once or twice daily when consuming the oral doses.

Although the present invention has been described above in detail, it will be understood that the full scope of the invention is not limited by the above description. Rather, the invention is defined by the claims of this patent specification, including all reasonable equivalents to any element defined therein.

1. A method for the treatment of aged, wrinkled or otherwise damaged skin by topical daily application of a combination of an anti-aging or anti-wrinkle topical formulation, once daily oral consumption of a first oral formulation and once daily oral consumption of a second oral formulation, said first and second formulations consumed approximately 12 hours apart, said first and second formulations each comprising one or more antioxidants and antioxidant co-promoters, anti-inflammatories, and collagen synthesis promoters.

2. A method as defined in claim 1, wherein antioxidants comprise one or more of: ginger, tumeric, green tea, alpha-lipoic acid, flavenoids, grape seed, blueberry, or selenium, or derivatives or extracts thereof.

3. A method as defined in claim 1, wherein said anti-inflammatory compounds are plant-derived.

4. A method as defined in claim 3, wherein said anti-inflammatory compounds are extracted or derived from one or both of ginger or tumeric.

5. A method as defined in claim 2, wherein said flavonoids comprise or are derived or extracted from one or more of quercetin, red wine, buckwheat or blueberry.

6. A method as defined in claim 1, wherein at least one of said first and second oral formulations includes at least one of the following: flax seed powder green tea, DMAE (as DMAE bitartrate), ginger root, alpha-lipoic acid, bioflavonoid complex, aloe vera leaf, n-acetyle 1-carnitine, 1-carnosine, grape seed, quercetin, red wine, blueberry, 1-proline, tumeric, hyaluronic acid, vitamin A, vitamin C, vitamin E, vitamin B-1, vitamin B-6, folic acid, potassium, calcium carbonate, magnesium citrate, manganese sulfate, jujub extract, magnolia bark or extract, conjugated lipoic acid, chamomile flower or extract, royal jelly, coenzyme Q-10, 1-lysine, pine bark or extract, reservatrol, RNA (ribonucleic acid), calcium, zinc, selenium and copper.

7. A method as defined in claim 1, wherein said first and second oral formulations are different from each other.

8. A method as defined in claim 7, wherein said first and second oral formulations contain antioxidants and antioxidant co-promoters, anti-inflammatories, vitamins, nutritionally desirable minerals, and collagen synthesis promoters all different from each other.

9. A method as defined in claim 1, wherein said anti-aging or anti-wrinkle topical formulation comprises a palmitol peptide, glucosamine and a palmitoyl oligopeptide.

10. A method as defined in claim 1, wherein at least one of said first and second oral formulations further comprise at least one nutritionally useful vitamin.

11. A method as defined in claim 1, wherein at least one of said first and second oral formulations comprises at least one vitamin.

12. A method as defined in claim 11, wherein said at least one vitamin comprises vitamin E.

13. A method as defined in claim 1, wherein said first oral formulation comprises: flax seed powder, green tea, DMAE, ginger root powder, alpha-lipoic acid, bioflavonoid complex, aloe vera leaf, n-acetyle 1-carnitine, 1-carnosine, grape seed, quercetin, red wine, blueberry extract, 1-proline, tumeric powder, hyaluronic acid, vitamin A, vitamin C, vitamin E, vitamin B-1, vitamin B-6, folic acid, potassium, calcium carbonate, magnesium citrate and manganese sulfate.

14. A method as defined in claim 1, wherein said second oral formulation comprises: jujub or extract, magnolia bark or extract, conjugated lipoic acid, chamomile flower, royal jelly, coenzyme Q-10, 1-lysine, pine bark or extract, reservatrol, RNA (ribonucleic acid), calcium, zinc, selenium and copper.

15. A method as defined in claim 1, wherein said first and second oral formulations include a stimulant and sedative respectively for morning and evening consumption respectively.

16. A kit for the treatment of aged, wrinkled or otherwise damaged skin by topical daily application of a combination of an anti-aging or anti-wrinkle topical formulation, once daily oral consumption of a first oral formulation and once daily oral consumption of a second oral formulation, said first and second formulations consumed approximately 12 hours apart, said first and second formulations each comprising one or more antioxidants and antioxidant co-promoters, anti-inflammatories, vitamins, nutritionally desirable minerals, and collagen synthesis promoters.

17. A kit as defined in claim 16, wherein antioxidants comprise one or more of: ginger, tumeric, green tea, alpha-lipoic acid, flavenoids, grape seed, blueberry, or selenium, or derivatives or extracts thereof.

18. A kit as defined in claim 16, wherein said anti-inflammatory compounds are plant-derived.

19. A kit as defined in claim 16, wherein said anti-inflammatory compounds are extracted or derived from one or both of ginger or tumeric.

20. A kit as defined in claim 17, wherein said flavonoids comprise or are derived or extracted from one or more of quercetin, red wine, buckwheat or blueberry.

21. A kit as defined in claim 16, wherein at least one of said first and second oral formulations includes at least one of the following: flax seed powder green tea, DMAE (as DMAE bitartrate), ginger root, alpha-lipoic acid, bioflavonoid complex, aloe vera leaf, n-acetyle 1-carnitine, 1-carnosine, grape seed, quercetin, red wine, blueberry, 1-proline, tumeric, hyaluronic acid, vitamin A, vitamin C, vitamin E, vitamin B-1, vitamin B-6, folic acid, potassium, calcium carbonate, magnesium citrate, manganese sulfate, jujub extract, magnolia bark or extract, conjugated lipoic acid, chamomile flower or extract, royal jelly, coenzyme Q-10, 1-lysine, pine bark or extract, reservatrol, RNA (ribonucleic acid), calcium, zinc, selenium and copper.

22. A kit as defined in claim 16, wherein said first and second oral formulations are different from each other.

23. A kit as defined in claim 22, wherein said first and second oral formulations contain different antioxidants and antioxidant co-promoters, anti-inflammatories, vitamins, nutritionally desirable minerals, and collagen synthesis promoters.

24. A kit as defined in claim 16, wherein said anti-aging or anti-wrinkle topical formulation comprises a palmitol peptide, glucosamine and a palmitoyl oligopeptide.

25. A kit as defined in claim 16, wherein at least one of said first and second oral formulations further comprise at least one nutritionally useful vitamin.

26. A kit as defined in claim 16, wherein at least one of said first and second oral formulations comprises at least one vitamin.

27. A kit as defined in claim 26, wherein said at least one vitamin comprises vitamin E.

28. A kit as defined in claim 16, wherein said first oral formulation comprises: flax seed powder, green tea, DMAE, ginger root powder, alpha-lipoic acid, bioflavonoid complex, aloe vera leaf, n-acetyle 1-carnitine, 1-carnosine, grape seed, quercetin, red wine, blueberry extract, 1-proline, tumeric powder, hyaluronic acid, vitamin A, vitamin C, vitamin E, vitamin B-1, vitamin B-6, folic acid, potassium, calcium carbonate, magnesium citrate and manganese sulfate.

29. A kit as defined in claim 16, wherein said second oral formulation comprises: jujub or extract, magnolia bark or extract, conjugated lipoic acid, chamomile flower, royal jelly, coenzyme Q-10, 1-lysine, pine bark or extract, reservatrol, RNA (ribonucleic acid), calcium, zinc, selenium and copper.

30. A kit as defined in claim 16, wherein said first and second oral formulations include a stimulant and sedative respectively for morning and evening consumption respectively.
